(19) United States
(12) Patent Application Publication
(10) Pub. No.: US 2012/0191189 A1
(43) Pub. Date: Jul. 26, 2012

(54) Spinal implant with padded bone engaging projections

(76) Inventor: Meng-Feng Huang, Changhua (TW)

(21) Appl. No.: 13/009,862

(22) Filed: Jan. 20, 2011

(51) Int. Cl. A61F 2/44

(52) U.S. Cl. 623/17.11

(57) ABSTRACT

A spinal implant in one embodiment includes an implant for insertion between two opposite spaced vertebrae of a spine, comprising an upper section having a substantially rectangular cross section and comprising a toothed top retaining member and a peripheral surface; a lower section having a substantially rectangular cross section and comprising a toothed bottom retaining member and a peripheral surface; an intermediate padding member for fastening the upper section and the lower section together; two three-dimensional matrix structures formed in the upper section and the lower section respectively and on the peripheral surfaces of the upper section and the lower section respectively as support; and a plurality of holes formed through at least one of three directions of each of the three-dimensional matrix structures.

### BACKGROUND OF THE INVENTION

1. Field of Invention

The invention relates to surgical procedures for stabilizing the spine and more particularly to an improved implant having padded bone engaging projections for use in such procedure.

2. Description of Related Art

In human anatomy, the vertebral column (backbone or spine) is a column usually consisting of 24 articulating vertebrae (including 7 vertebrae in cervical region, 12 vertebrae in thoracic region, and 5 vertebrae in lumbar region) and 9 fused vertebrae in the sacrum and the coccyx. It is situated in the dorsal aspect of the torso, separated by intervertebral discs. It houses and protects the spinal cord in its spinal canal.

Intervertebral discs lie between adjacent vertebrae in the spine. Each intervertebral disc forms a cartilaginous joint to allow slight movement of the vertebrae, and acts as a ligament to hold the vertebrae together. An intervertebral disc consists of an outer annulus fibrosus surrounding the inner nucleus pulposus. The annulus fibrosus consists of several layers of fibrocartilage. The strong annular fibers contain the nucleus pulposus and distribute pressure evenly across the disc. The nucleus pulposus contains loose fibers suspended in a mucoprotein gel with the consistency of jelly. The nucleus of the intervertebral disc acts as a shock absorber, absorbing the impact of the body's daily activities and keeping the two vertebrae separated.

Chronic low back pain is a perplexing problem facing the field of orthopedic surgery. Low back pain can be avoided by preventing relative motion between spinal vertebrae (commonly known as intervertebral stabilization). To abate low back pain, stabilization is directed to stabilizing contiguous vertebrae in the lumbar region of the spine. Surgical techniques seek to rigidly join vertebrae which are separated by a degenerated disc. One typical technique is to partially remove a degenerated disc and to insert a bone graft into the void formed by the removed disc. Spinal implants are also employed and are either acting along or in combination with bone fragments to replace the use of bone grafts.

However, improvements of spinal implant are still desired in order to enhance patient safety and the probability of a satisfactory recovery.

### SUMMARY OF THE INVENTION

It is therefore one object of the invention to provide an implant for insertion between two opposite spaced vertebrae of a spine, comprising an upper section having a substantially rectangular cross section and comprising a toothed top retaining member and a peripheral surface; a lower section having a substantially rectangular cross section and comprising a toothed bottom retaining member and a peripheral surface; an intermediate padding member for fastening the upper section and the lower section together; two three-dimensional matrix structures formed in the upper section and the lower section respectively and on the peripheral surfaces of the upper section and the lower section respectively as support; and a plurality of holes formed through at least one of three directions of each of the three-dimensional matrix structures.

It is another object of the invention to provide an implant for insertion between two opposite spaced vertebrae of a spine, comprising an upper section having a substantially rectangular cross section and comprising a toothed top retaining member and a peripheral surface; a lower section having a substantially rectangular cross section and comprising a toothed bottom retaining member and a peripheral surface; an intermediate padding member for fastening the upper section and the lower section together; two three-dimensional matrix structures formed in the upper section and the lower section respectively and on the peripheral surfaces of the upper section and the lower section respectively as support; a plurality of holes formed through at least one of three directions of each of the three-dimensional matrix structures; a longitudinal channel formed in central portions of each of the three-dimensional matrix structures and the padding member and communicating with the holes; and an elastic device disposed in the channel.

The above and other objects, features and advantages of the invention will become apparent from the following detailed description taken with the accompanying drawings.

### BRIEF DESCRIPTION OF THE DRAWINGS

FIG. 1 is a perspective view of a spinal implant according to a first preferred embodiment of the invention;

FIGS. 2 and 3 are sectional views taken along line 2-2 and line 3-3 of FIG. 1 respectively;

FIG. 4 schematically depicts a fixing of the spinal implant in a bore formed between opposing vertebrae of a spine;

FIG. 5 is a perspective view of a spinal implant according to a second preferred embodiment of the invention;

FIGS. 6 and 7 are sectional views taken along line 6-6 and line 7-7 of FIG. 5 respectively;

FIG. 8 schematically depicts a fixing of the spinal implant of FIG. 5 in a bore formed between opposing vertebrae of a spine;

FIG. 9 is a perspective view of a spinal implant according to a third preferred embodiment of the invention; and

FIG. 10 is a perspective view of a spinal implant according to a fourth preferred embodiment of the invention.

### DETAILED DESCRIPTION OF THE INVENTION

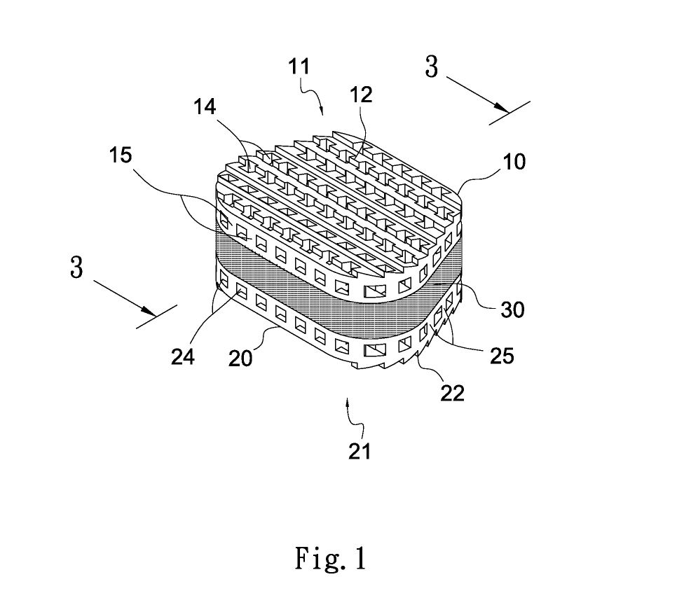

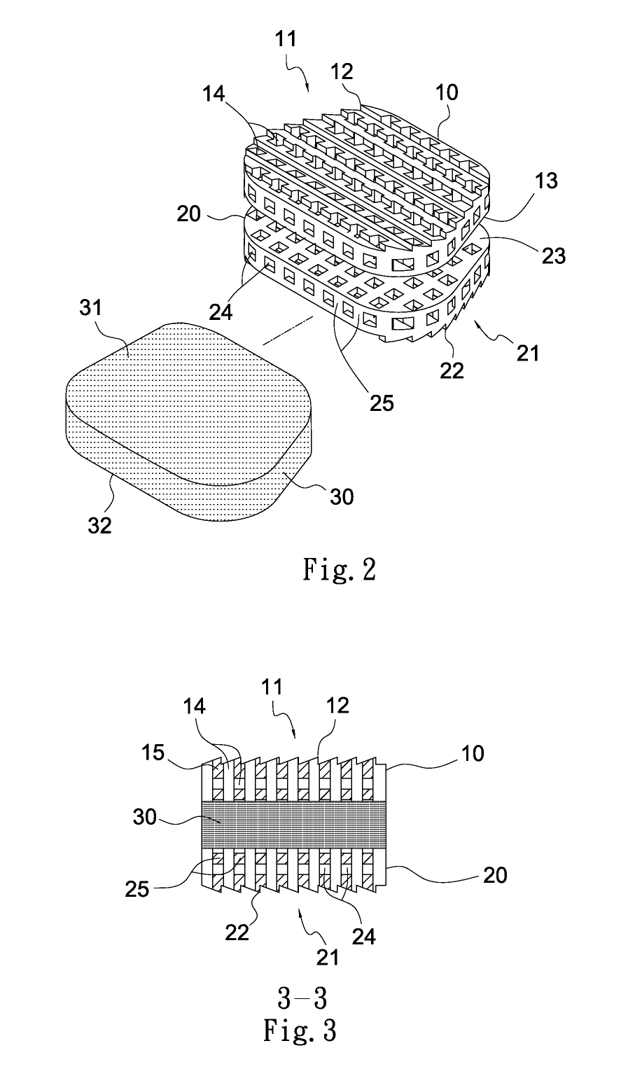

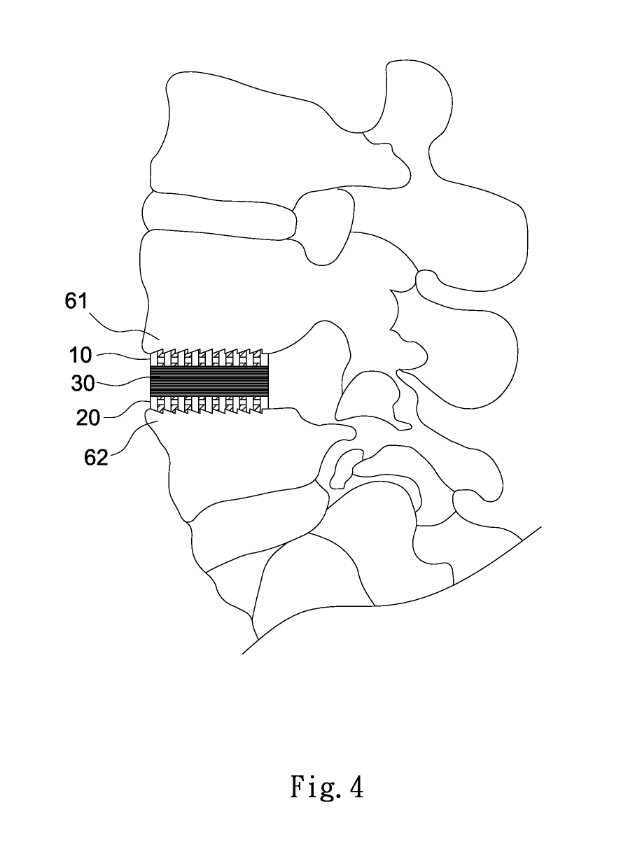

Referring to FIGS. 1 to 4, a spinal implant in accordance with a first preferred embodiment of the invention is shown. The spinal implant has a substantially rectangular cross section and comprises the following components as discussed in detail below.

An upper section 10 is formed of a composite material being sturdy and highly resistant to chemicals. The composite material may be carbon fiber or PEEK (polyetheretherketone). Alternatively, the upper section 10 is formed of alloy such as stainless steel, cobalt-chromium-molybdenum alloy, titanium, or titanium alloy. Still alternatively, the upper section 10 is formed of polymer such as UHMWPE (ultra high molecular weight polyethylene), PMMA (polymethylmethacrylate), silicon rubber, or ultra high molecular polyethylene. Still alternatively, the upper section 10 is formed of ceramic such as aluminum oxide, calcium phosphate tri-basic, or fiber glass.

The upper section 10 has a top surface 11 with a top retaining member 12 formed thereon. The top retaining member 12 is shaped as a plurality of rows of teeth (i.e., bone engaging projections), an engaging bottom 13, and a three-dimensional matrix structure 15 formed between the top surface 11 and the engaging bottom 13 as support. The three-dimensional matrix structure 15 can provide mechanical properties such as enhanced resistance to pressure, enhanced resistance to stress, and enhanced resistance to tension to the upper section 10. A plurality of holes 14 are formed through each of three directions of the three-dimensional matrix structure 15. The holes 14 occupy about 1% to 90% of the volume of the upper section 10 depending upon engineering choice of design. Each hole 14 has a bore of about 150 μm to 1,000 μm.

A lower section 20 is formed of a composite material being sturdy and highly resistant to chemicals. The composite material may be carbon fiber or PEEK (polyetheretherketone). Alternatively, the lower section 20 is formed of alloy such as stainless steel, cobalt-chromium-molybdenum alloy, titanium, or titanium alloy. Still alternatively, the lower section 20 is formed of polymer such as UHMWPE (ultra high molecular weight polyethylene), PMMA (polymethylmethacrylate), silicon rubber, or ultra high molecular polyethylene. Still alternatively, the lower section 20 is formed of ceramic such as aluminum oxide, calcium phosphate tri-basic, or fiber glass.

The lower section 20 is an inverted mirror image of the upper section 10 and comprises a bottom surface 21 with a bottom retaining member 22 formed thereon. The bottom retaining member 22 is shaped as a plurality of rows of teeth (i.e., bone engaging projections). The lower section 20 further comprises an engaging top 23 and a supporting three-dimensional matrix structure 25 formed between the bottom surface 21 and the engaging top 23. The three-dimensional matrix structure 25 can provide mechanical properties such as enhanced resistance to pressure, enhanced resistance to stress, and enhanced resistance to tension to the lower section 20. A plurality of holes 24 are formed through each of three directions of the three-dimensional matrix structure 25. The holes 24 occupy about 1% to 90% of the volume of the lower section 20 depending upon engineering choice of design. Each hole 24 has a bore of about 150 μm to 1,000 μm.

A padding member 30 is formed of silicon rubber in this embodiment. The padding member 30 has excellent properties including great flexibility, oxidation resistant, corrosion proof, and water resistant. The padding member 30 comprises an engaging top 31 and an engaging bottom 32. The engaging top and bottom 31, 32 are permanently secured to the upper section 10 and the lower section 20 respectively by subjecting to heat.

As shown in FIG. 4 specifically, a physician may insert the spinal implant into a bore formed between an upper vertebra 61 and a lower vertebra 62 of a spine. The teeth of the top retaining member 12 and the teeth of the bottom retaining member 22 thus grasp the upper vertebra 61 and the lower vertebra 62 respectively (i.e., the spinal implant being fastened). Moreover, an appropriate biocompatible material may filled in the voids of the holes 14 and 24 for stuffing and stabilization purposes. The biocompatible material may be calcium phosphate tri-basic (CaP) or hydroxyapattie (HA) (Ca₁₀(PO₄)₆(OH)₂).

The holes 14 and 24 allow body tissues to grow therein for the health of spinal bone. Moreover, the fastening of the upper and lower vertebrae 61, 62 and the top and bottom retaining members 12, 22 is reliable so as to rigidly join the upper and lower vertebrae 61, 62. As a result, intervertebral stabilization is carried out.

In addition, the provision of the three-dimensional matrix structures 15 and 25 can significantly increase resistance to pressure, stress, and tension to the spinal implant. Moreover, the padding member 30 acts as a shock absorbing and buffering device. Therefore, the spinal implant is sturdy and a useful life of the spinal implant can be prolonged.

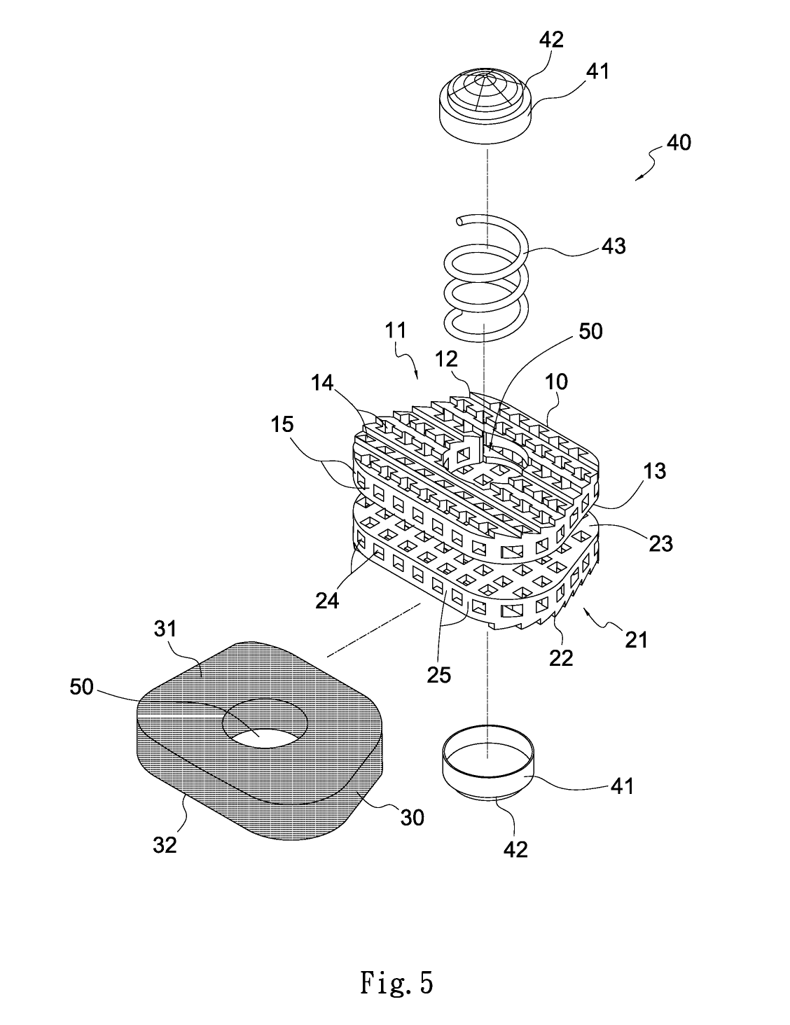

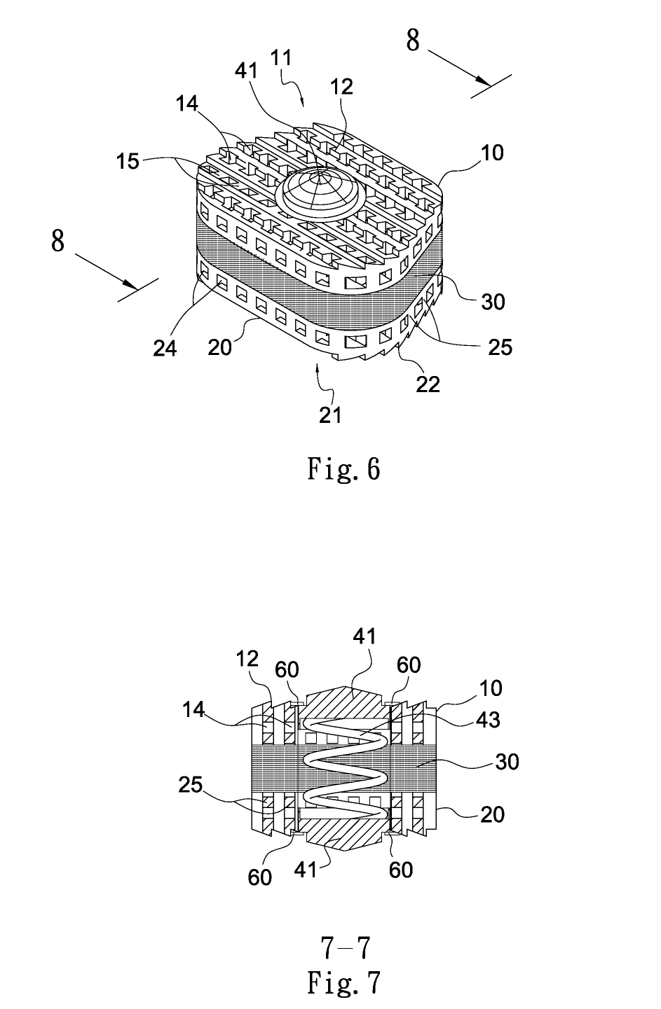

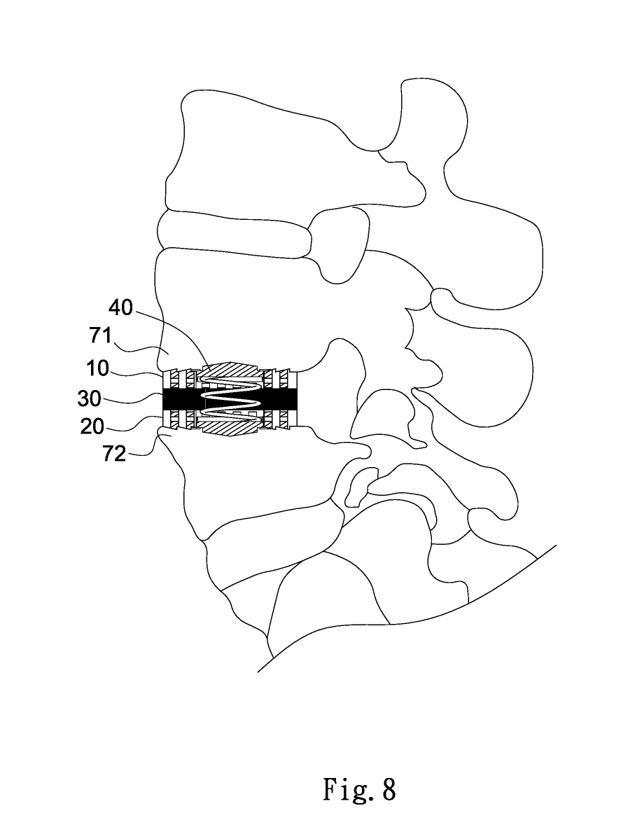

Referring to FIGS. 5 to 8, a spinal implant in accordance with a second preferred embodiment of the invention is shown. The characteristics of the second preferred embodiment are substantially the same as that of the first preferred embodiment except the following: A longitudinal channel 50 of circular section is formed in central portions of the upper section 10, the padding member 30, and the lower section 20 and communicates with the holes 14 and 24. An elastic device 40 is provided in the channel 50 and comprises a helical spring 43, two cup-shaped caps 41 at both ends of the spring 43, and the cap 41 having a peripheral shoulder 42 on an outer surface. Two stop members 60 each is provided on the top surface 11 of the upper section 10 or the bottom surface 21 of the lower section 20. The stop members 60 are adapted to prevent from the caps 41 from being disengaging from the spring 43 when the caps 41 contact the stop members 60 due to expansion of the spring 43. The provision of the elastic device 40 further increases the shock absorbing and buffering capabilities of the spinal implant.

After inserting the spinal implant between the upper and lower vertebrae 71, 72 with the upper and lower vertebrae 71, 72 and the top and bottom retaining members 12, 22 being fastened together in a surgery, bone tissues may grow to fill in the holes 14, 24. This has the benefits of carrying out intervertebral stabilization and increasing the probability of a satisfactory recovery of a patient.

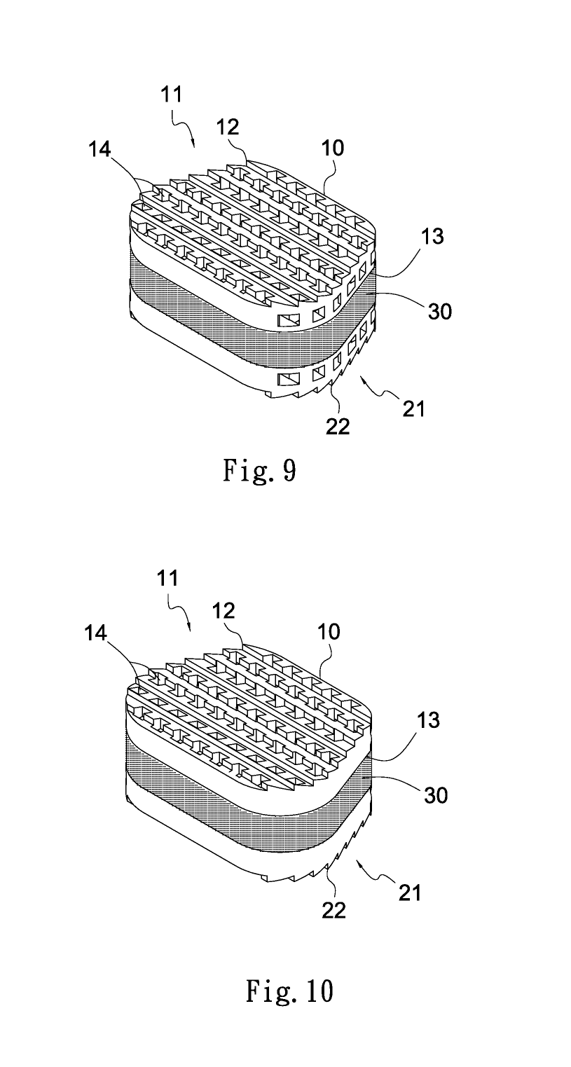

Referring to FIG. 9, a spinal implant in accordance with a third preferred embodiment of the invention is shown. The characteristics of the third preferred embodiment are substantially the same as that of the first preferred embodiment except the following: The peripheral surface of each of the upper section 10 and the lower section 20 has two relatively smooth, opposite sides. No holes are formed along the direction of the opposite sides of the peripheral surface of each of the upper section 10 and the lower section 20.

Referring to FIG. 10, a spinal implant in accordance with a fourth preferred embodiment of the invention is shown. The characteristics of the fourth preferred embodiment are substantially the same as that of the first preferred embodiment except the following: The peripheral surface of each of the upper section 10 and the lower section 20 has all of four sides being relatively smooth. No holes are formed transversely in each of the upper section 10 and the lower section 20 (i.e., both the upper section 10 and the lower section 20 having no transverse holes).

While the invention has been described in terms of preferred embodiments, those skilled in the art will recognize that the invention can be practiced with modifications within the spirit and scope of the appended claims.

What is claimed is:

1. An implant for insertion between two opposite spaced vertebrae of a spine, comprising:
an upper section having a substantially rectangular cross section and comprising a toothed top retaining member and a peripheral surface;
a lower section having a substantially rectangular cross section and comprising a toothed bottom retaining member and a peripheral surface;
an intermediate padding member for fastening the upper section and the lower section together;
two three-dimensional matrix structures formed in the upper section and the lower section respectively and on the peripheral surfaces of the upper section and the lower section respectively as support; and
a plurality of holes formed through at least one of three directions of each of the three-dimensional matrix structures.

2. The spinal implant of claim 1, wherein the holes are formed through one transverse direction of the three directions of each of the three-dimensional matrix structures.

3. The spinal implant of claim 1, wherein the holes are formed through two transverse directions of the three directions of each of the three-dimensional matrix structures.

4. The spinal implant of claim 1, wherein the holes are formed through each of the three directions of each of the three-dimensional matrix structures.

5. The spinal implant of claim 1, wherein the holes occupy about 1% to 90% of a volume of each of the three-dimensional matrix structures, and wherein each hole has a bore of about 150 μm to 1,000 μm.

6. An implant for insertion between two opposite spaced vertebrae of a spine, comprising:
an upper section having a substantially rectangular cross section and comprising a toothed top retaining member and a peripheral surface;
a lower section having a substantially rectangular cross section and comprising a toothed bottom retaining member and a peripheral surface;
an intermediate padding member for fastening the upper section and the lower section together;
two three-dimensional matrix structures formed in the upper section and the lower section respectively and on the peripheral surfaces of the upper section and the lower section respectively as support;
a plurality of holes formed through at least one of three directions of each of the three-dimensional matrix structures;
a longitudinal channel formed in central portions of each of the three-dimensional matrix structures and the padding member and communicating with the holes; and
an elastic device disposed in the channel.

7. The spinal implant of claim 6, wherein the holes are formed through one transverse direction of the three directions of each of the three-dimensional matrix structures.

8. The spinal implant of claim 6, wherein the holes are formed through two transverse directions of the three directions of each of the three-dimensional matrix structures.

9. The spinal implant of claim 6, wherein the holes are formed through each of the three directions of each of the three-dimensional matrix structures.

10. The spinal implant of claim 6, wherein the holes occupy about 1% to 90% of a volume of each of the three-dimensional matrix structures, and wherein each hole has a bore of about 150 μm to 1,000 μm.

11. The spinal implant of claim 6, wherein the elastic device comprises a biasing member and two caps at both ends of the biasing member, the caps being on top of the upper section and bottom of the lower section respectively.

12. The spinal implant of claim 11, wherein each of the caps has a peripheral shoulder on an outer surface, and further comprising two stop members each disposed on the top of the upper section or the bottom of the lower section, the stop member being adapted to limit a travel distance of each of the caps when they are engaged.
